(19) United States
(12) Patent Application Publication
(10) Pub. No.: US 2008/0254090 A1
(43) Pub. Date: Oct. 16, 2008

(54) Porcine Islets Cultured With Porcine Sertoli Cells For Xenotransplantation

(75) Inventors: Stephen John Martin Skinner, Auckland (NZ); Robert Bartlett Elliott, Auckland (NZ); Livia Del Carmen Escobar Orellana, Auckland (NZ)

Correspondence Address: Mark D Moore;Haynes and Boone, Suite 3100, 901 Main Street, Dallas, TX, 75202-3789, US

(73) Assignee: DIABCELL PTY LIMITED, PARKSIDE, SOUTH AUSTRALIA (AU)

(21) Appl. No.: 10/562,246

(22) PCT Filed: Jun. 24, 2004
(86) PCT No.: PCT/NZ03/00130

§ 371 Date: Apr. 23, 2007

(51) Int. Cl. A61K 35/12; C12N 5/02; C12N 5/06; A61K 9/00; A61P 3/10

(52) U.S. Cl. 424/423; 435/373; 435/347; 424/93.3

(57) ABSTRACT

Aggregates and their method of preparation suitable for implantation into a recipient in order to produce insulin in vivo. The methods involve culturing islet cells isolated from the pancreas of donor piglets with isolated Sertoli cells from the testes of donor piglets. A preferred period of culturing is 5 days and may be followed by a purification procedure.

### TECHNICAL FIELD

The invention relates to the use of porcine pancreatic islet cells for the treatment of diabetes.

More particularly but not exclusively it relates to the use of porcine pancreatic islet cells with associated Sertoli cells for the treatment of diabetes by xenotransplantation.

### BACKGROUND

### Background and Rationale for Porcine Islet Cell Xenotransplantation.

Type 1 (insulin-dependent) diabetes mellitus is a common endocrine disorder that results in substantial morbidity and mortality, and has a major financial impact on individual patients and healthcare systems. Treatment with insulin, while life-saving, often does not provide sufficient control of blood glucose to prevent the life-shortening complications of the disease, and this has given rise to intensive research into better methods of achieving and sustaining normoglycaemia. Among the newer treatment strategies that have been proposed, transplantation of pancreatic β islet cells, obtained either from other humans or animals, has received the most attention worldwide. This is because islet cell transplantation can restore not only the insulin-secreting unit, but also the precise fine-tuning of insulin release in response to multiple neural and humoral signals arising within and beyond the islets of Langerhans.

As human islet cell transplantation (allotransplantation) is limited by the shortage of human islet tissue, the use of pig islet cells is currently viewed as the most promising alternative since:
(a) pig and human insulin have close structural and biological similarities;
(b) physiological glucose levels in pigs are similar to those in humans; and
(c) the supply of pig cells can be readily expanded by optimising the supply of donor animals.

The rationale for this treatment approach (termed ‘xenotransplantation’) is that the implanted porcine islets have the potential to mimic the normal physiological insulin response in type 1 diabetics, such that near-normal blood glucose levels may be achievable without insulin or with a reduced requirement for it. As a consequence, long-term diabetes complications may be prevented and patients should experience less hypoglycaemia than they do with the currently recommended ‘intensive’ insulin regimens.

### Barriers to the Introduction of Porcine Islet Cell Xenotransplantation and Measures Adopted to Address Them

Any new treatment strategy is burdened with problems and pitfalls before it can be implemented, and xenotransplantation of porcine islet cells is no exception. There have been a number of scientific and ethical/political barriers to implementation of the procedure, but as knowledge in the field has grown, these barriers have steadily receded. The problems that have arisen include:
1. Rejection of islet cells by the recipient's immune system: the vulnerability of the transplanted islets to the recipient's immune system has been a major scientific barrier to successful islet cell transplantation. Strategies employed to address the problem include:
   a) Concurrent administration of immunosuppressive drugs—which, though successfully utilised in some recent allotransplantation studies, has the dual disadvantage of producing adverse effects on both the transplanted islets (i.e. impairing their engraftment and function and reducing their insulin secretory responses) and the recipient (i.e. exposing patients to the risks of a variety of serious complications, including nephrotoxicity, neurotoxicity, hypertension, increased susceptibility to infection and osteoporosis). Moreover, this approach is not always effective in altering the course and incidence of rejection episodes.
   b) Development of novel non-drug ‘immunoprotection’ strategies to shield the transplanted islets from the recipient's immune system and thus prevent local inflammatory responses and chronic rejection, while still allowing them to function by secreting insulin and controlling glucose metabolism in the body. Among the various ‘immunoprotection’ strategies that have been investigated are:
      Tubular diffusion chambers and perfusion devices (artificial pancreases). As yet there is little evidence that this approach is clinically useful.
      Encapsulation of the transplanted islets in alginate microcapsules. This approach, which we have previously shown in experimental studies to confer significantly longer functional durations on transplanted islets in comparison with unencapsulated islets, has been extensively investigated in experimental models of diabetes. Reversal of the diabetic state with xenotransplants of alginate-encapsulated porcine islets has been accomplished in CD1 mice rendered diabetic by streptozotocin, NOD (non-obese diabetic) mice, New Zealand white rabbits rendered diabetic by alloxan, and a spontaneously diabetic dog. In addition, a preliminary clinical study with alginate-encapsulated porcine islets undertaken in two type 1 diabetic patients has provided encouraging results, with both patients exhibiting reduced insulin requirements. Continued functioning of the islets was still evident in both individuals at 14 months after transplantation with no evidence of adverse effects or any evidence of porcine retroviral infection.
      Cotransplantation of islets with Sertoli cells isolated from the testes of male donor animals. This approach has been shown to protect islets against immune-mediated rejection and enhance their functional performance and longevity.
2. Possible transmission of infectious diseases: this potential concern centres around the risk of transmission of porcine diseases to the recipient, and the risk of introducing micro-organisms during cell processing.
3. Ethical issues concerning xenotransplantation: these include concerns over the ethical acceptability of using animal tissues for transplantation, the welfare of donor animals, obtaining informed consent from patients selected for clinical trials, and the impact of the procedure on them. These issues have been addressed by bodies such as the Nuffield Council on Bioethics in the UK and a number of recommendations to protect the ethical integrity of future human research have been made. These include the “ethical acceptability” of using porcine tissues for xenotransplantation; the need to avoid or minimise harm to donor animals; the requirement to provide patients with a detailed explanation of the likely success, attendant risks, and the subsequent quality-of-life that can be expected when obtaining their informed consent; and informing patients that their consent to the procedure includes consent to ongoing post-transplant microbiological monitoring.

### OBJECT OF THE INVENTION

It is an object of the invention to provide a method of treatment of diabetes, and/or a means to aid treatment of diabetes which has improvements to, or provides an alternative from, the abovementioned methods and/or means.

### STATEMENTS OF THE INVENTION

According to a first aspect of the invention there is provided a method of preparing aggregates of porcine pancreatic islets and porcine Sertoli cells capable upon implantation into a recipient, of producing insulin in vivo, including or comprising the steps of:
1) isolation of porcine islet cells from the pancreas of donor piglets,
2) isolation of porcine Sertoli cells from the testes of donor piglets,
3) culturing the islet cells together with the Sertoli cells,
4) formation of the aggregates.

Preferably the combination is in a predetermining ratio from 1:20,000 (islet:Sertoli cells) to 1:100; more preferably the ratio is between 1:2,000 to 1:4,000.

Preferably the culturing step is over a time period between 3 to 7 days more preferably it is for 5 days.

Preferably the isolation of the islets is followed by purification of the islets.

Preferably the isolation and purification of the islets together comprise or include the steps of:
a) surgical removal,
b) collagenase digestion,
c) washing and culturing of the islets.

Preferably the digestion involves Liberase H and Xylocaine.

Preferably the isolation of the Sertoli cells is followed by purification of the Sertoli cells.

Preferably the isolation and purification of the Sertoli cells together comprise or include the steps of:
a) surgical removal,
b) digestion with trypsin, Dnase,
c) washing and culturing of the cells.

Preferably the method includes the step
5) virological and microbiological testing and/or monitoring of the aggregates and/or components thereof.

Preferably or alternatively the method includes a prestep (before step 1)) of virological monitoring and/or testing of one or preferably both of the islets and Sertoli cells.

Preferably the method includes additionally or alternatively a pre-step of virological monitoring and/or testing of the piglet donors.

Preferably the islets and Sertoli cells derive from the same herd, more preferably from the same donor piglet.

Preferably the piglets are one week old donors.

Preferably the piglets are monitored and/or tested for infectious agents.

Preferably the pig herd is a New Zealand pig herd.

Preferably the step of the formation of the aggregates involves: the preservations of the original characteristics and/or native structure of the islets.

According to a further aspect of the invention there is provided an aggregate of porcine islets with Sertoli cells prepared substantially according to the above method.

According to a third aspect of the invention there is provided a method of treating a patient suffering from diabetes mellitus comprising or including the steps of:
1) preparing one or more aggregates of porcine islets with Sertoli cells prepared substantially according to the above method,
2) implanting or otherwise administering one or more aggregate to the patient.

Preferably the step of implanting or administering the aggregate may be by:
encapsulation of the aggregate in a suitable biocompatible material (more preferably a suitable alginate),
confinement into a suitable device (more preferably a vascularized tube for example)
matrix preparations including preparation of gelatin, collagen, and natural carbohydrate polymers.
plasma thrombin clot—autologous plasma clots produced with allogeneic thrombin.

According to a further aspect of the invention there is provided a device for implantation into a recipient suffering from diabetes mellitus, the device incorporating aggregates of porcine pancreatic islets and porcine Sertoli cells, the aggregates being, or possessing the characteristics of, the aggregates previously described.

Preferably the device incorporating the aggregates may be one of:
a suitable biocompatible material as a capsule (more preferably of a suitable alginate);
a vascularized tube;
a matrix preparation including preparation of gelatin, collagen, and natural carbohydrate polymers.
a plasma thrombin clot—autologous plasma clots produced with allogeneic thrombin.

According to a further aspect of the invention there is provided a method of preparing aggregates of porcine pancreatic islets and porcine Sertoli cells prepared substantially according to FIG. 1.

According to a further aspect of the invention there is provided an aggregate of porcine pancreatic islets and porcine Sertoli cells substantially as described herein and with reference to any one or more of FIGS. 2 to 5.

### DESCRIPTION OF THE FIGURES

FIG. 1 illustrates a flow diagram of the preferred method of aggregate preparation according to the invention;

FIGS. 2-5 illustrate islet-sertoli cell aggregates of the invention.

### DETAILED DESCRIPTION

The invention disclosed herein relates to the preparation and use of an “aggregate” of Sertoli cells with porcine islets.

Prior art methods involving the use of Sertoli cells and islets (or other cells) have generally involved processing and isolation of each separately and putting together at the time of the transplant.

We have found that preparation of an aggregate, in a predetermining ratio of Sertoli to islet cells, and co-culturing allows the islets time to grow and to use the growth factors deemed from the Sertoli cells in vitro before the transplant. We have found the islets function better as they are protected by the layer of Sertoli cells.

Ideally both the islets and Sertolis are derived from the same donors. This simplifies viral screening.

By “aggregate” as used herein we specifically mean a discontinuous layer of Sertoli cells over the surface of the natural islet structure.

Rationale for Cotransplantation of Sertoli Cells with Islets

Cotransplantation of islet cells with Sertoli cells isolated from the testes of donor animals has been investigated as a means of achieving:
(a) protection against immune rejection; and
(b) stimulation of the mitotic rate of islet cells such that they release higher amounts of insulin in response to glucose stimulation and survive longer.

Sertoli cells are known to play a critical role in various physiological activities such as the synthesis of certain growth factors [e.g. insulin-like growth factors 1 and 2 (IGF-1, IGF-2) and epidermal growth factor (EGF)], immunomodulation [possibly as a result of increased secretion of transforming growth factor-beta 1 (TGF-β1)], and an anti-apoptotic (cell death inhibitory) function.

Our recent studies in experimental animal models have shown that the presence of Sertoli cells improves the in vitro functional competence of islets, and that xenotransplantation of islet-sertoli cell aggregates in diabetic rats, rabbits and NOD mice prolongs islet cell survival, leading to reversal of the diabetic state. The precise mechanism by which Sertoli cells protect islet cell grafts against immune rejection is not precisely known, but appears to be related to stimulation of the production of growth and differentiation factors by Sertoli cells.

Thus, our invention deals with cotransplantation of Sertoli cells with islets as aggregated such that the Sertoli cells can act as “nursing” cell systems for the islets, providing both efficient immunoprotection and enhancement of their functional performance and longevity.

This approach is complementary to, and synergistic with, other approaches for providing immunoprotection and functional longevity for transplanted islet cells.

In particular our invention deals with the use of islet-sertoli cell aggregates in:
Alginate-encapsulated form—to provide additional immune protection of the transplanted islets. The feasibility of co-microencapsulating Sertoli cells with islets isolated from rats has been demonstrated in our studies. We have, in our laboratories investigated the efficiency and safety of intraperitoncal transplants of alginate encapsulated Sertoli-islet cell aggregates in experimental animals.
Subcutaneous implant devices that allow the development of a prevascularised autologous collagen reservoir for the placement of the islet-sertoli cell aggregates. This approach is already being dealt with clinically in patients with type 1 diabetes mellitus.
Matrix preparations—in which islet-sertoli cell aggregates are cultured on gelatin, collagen and/or other matrices supplemented with natural carbohydrate polymers. Studies with this approach are currently being undertaken in animals with transplants of islet-sertoli cell aggregates with islet-sertoli cell ratios between 1:2,000 and 1:4,000.
Plasma Thrombin Clot—Autologous plasma clots produced with allogeneic thrombin as a biocompatible containment device.

We have determined, the islet cell: Sertoli cell ratio that provides optimal protection of the islets against immune rejection and maximal functional longevity may range from 1:20,000 ratio to provide maximal insulin release down to at least 1:2,000. The range is based on the findings of experimental studies with islet-Sertoli cell aggregates conducted in our laboratory, and in collaboration with the University of Perugia and National University of Singapore.

Preparation of our preferred Islet-Sertoli Cell Aggregates, in the Ratio of 1:2,000-1:4,000

The pig herd from which porcine islets and Sertoli cells for incorporation in our islet-sertoli cell aggregates are obtained comprises specific pathogen-free (SPF) NZ Large White pigs raised under strict biosecurity. Possible sources of zoonotic infections are monitored in the herd, the sows one month before farrowing, the donor piglets, and the tissue used. New Zealand is free from prion-mediated disease and many of the viral infections found in herds elsewhere in the world.

It would be envisaged by those skilled in the art that other suitable pig herds may be used if bred under suitable conditions, elsewhere in the world.

The islet cells are isolated from the pancreases of 7-day-old piglets via a major modification of the standard (Ricordi's) collagenase digestion procedure. All surgical procedures and cell processing are carried out with strict aseptic precautions. Following their isolation and purification, the islets are placed into culture tissue in RPMI medium enriched with 2% human serum albumin and 10 mmol/L nicotinamide. Culture at 37° C. in an air/5% CO₂ mixture with frequent changes of medium is then performed for 48 hours.

Sertoli cells are isolated from testicular cells of male 7-day-old piglets using a standard (Rajotte's) isolation method with modifications to ensure maximal cell yield. Following a number of quality control tests of both the islets and Sertoli cells (to ensure their optimal purity, viability and freedom from microbiological contamination; see further below), both the Sertoli cells and islets are counted and the latter adjusted to islets equivalents (IEQs) of 150 μm in diameter. The Sertoli cells are then combined with the islets in a ratio of 1:2,000-1:4,000, cultured for 24 hours, and scraped to form aggregates. Following a further 24 hours in culture, the islet-Sertoli cell aggregates are then tested for viability and insulin secretory capacity before being released for transplantation.

The production process for our islet-Sertoli aggregates preferably includes rigorous infection surveillance procedures comprising virological monitoring (see further below), screening for bacterial, fungal and mycoplasmal organisms, and bacterial endotoxin testing (LAL test). The presence of either microbiological contamination or a failure of the cells to meet any of the rigid quality control criteria set by the Applicant will lead to the particular cell batch being discarded.

### Aggregate Preparation

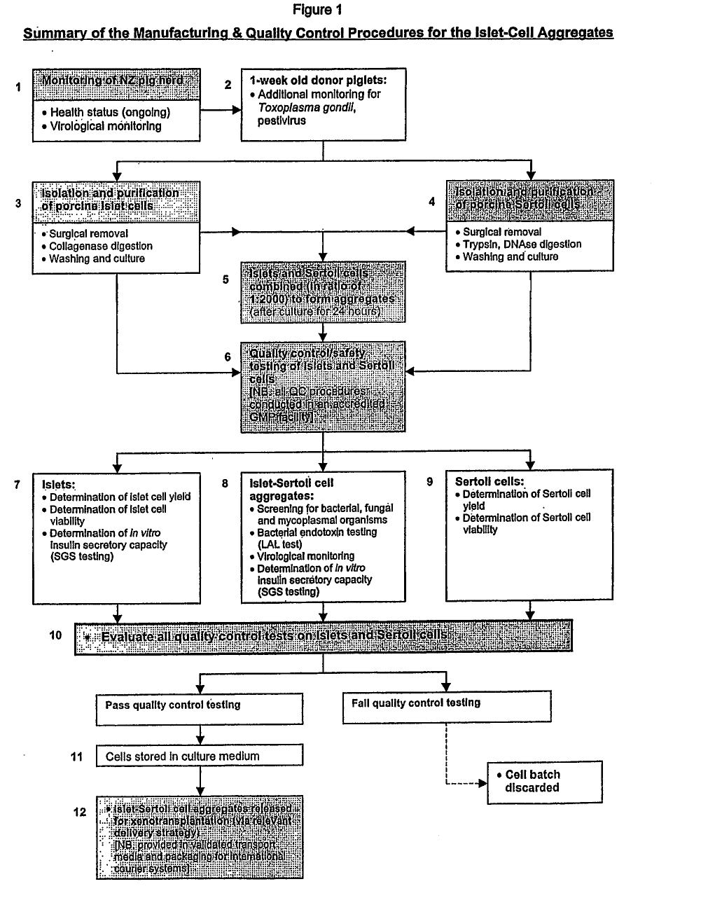

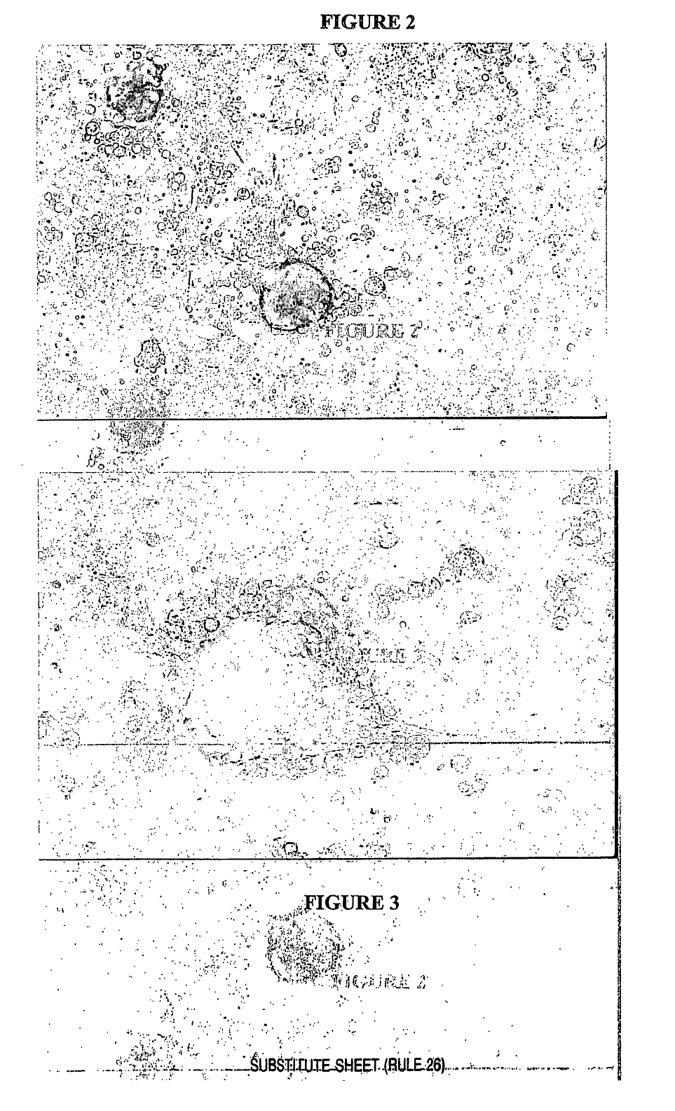

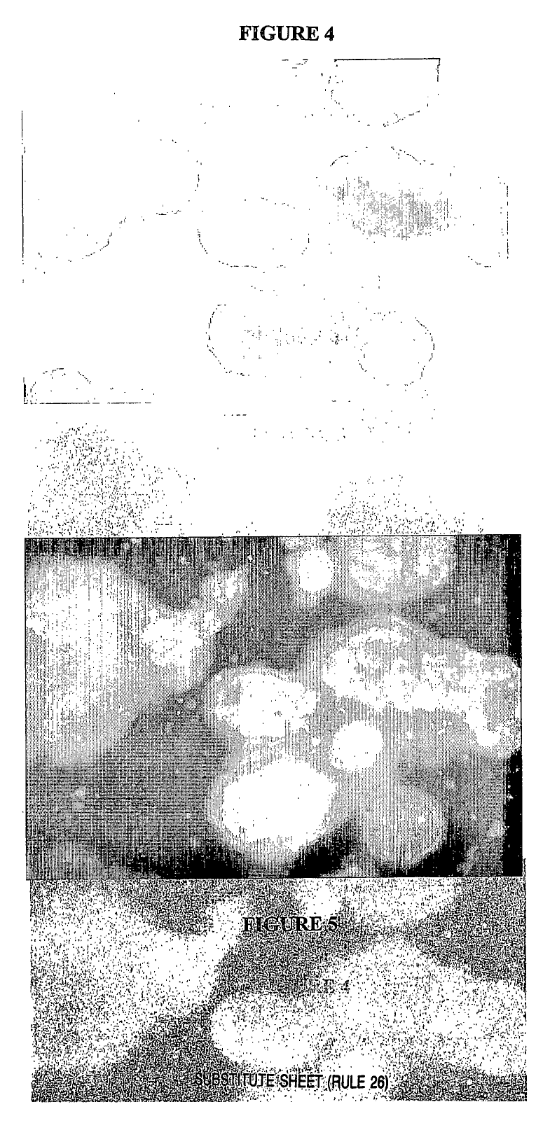

FIG. 1 illustrates a flow diagram of the preferred preparation method, and FIGS. 2-5 illustrate aggregates prepared by this method. Specifically FIG. 2 illustrates aggregates of 3 days, in culture (no staining, ×10); FIG. 3 illustrates aggregates of 3 days, in culture (no staining, ×20); FIG. 4 illustrates aggregates of 6 days, in culture (DTZ staining; purity >85%, 10×) and FIG. 5 illustrates aggregates of 6 days, in culture (AO/PI staining, viability >95%, 10×).

### 1) Sertoli Cells:

a) Introduction of Sertoli Cells
the testes are removed under sterile conditions from the donor,
the glands are minced into small pieces (approx. 1 mm each)
the minced tissue is washed twice with HBSS by sedimentation to eliminate red cells

b) First digestion of Sertoli Cells
the minced tissue is placed in 40 ml of digestion solution
Hanks with Calcium and Magnesium is added with Human Serum Albumin, Liberase H and Lignocaine
the bottle is held in a water bath at 37° C. for 18-20 minutes at 120 rpm
the tissue is washed 3 times with Hanks and centrifuged at 4° C. for 10 minutes at 1500 rpm.

c) Second digestion
Trypsin and Dnase are added
incubation at 37° C., 120 rpm until a white aggregate is produced.
the white aggregate is removed
the cells are seeded into petri dishes.

### 2) Pancreatic Islet Cells:

Are prepared according to our previously published method in WO 01/52871 (the contents of which are incorporated herein by reference).

3) Sertoli islet Aggregates:
After 1 day in culture the plates are washed and the islets (10,000 IEQ) added per plate. Ratio 1 islet:2,000 Sertoli cells.
Culture for 24 hours.
the cells are scraped and overlaid on the islets to form aggregates, then left for 24 hours in culture.
After this time the islet/Sertoli aggregates are ready for transplant or for encapsulation.
Staining with Trypan blue, SudanIII and Inhibin for sertoli cell viability and count

### Virological Monitoring

As indicated above, testing of the transplant material for the presence of PERV (porcine endogenous retrovirus), using a highly specific and highly sensitive assay developed for this purpose, is preferably an integral part of our islet-sertoli cell aggregate production process. In addition to PERV, attention is also directed towards other potentially infectious pathogens that can cause zoonoses and xenoses, including porcine cytomegalovirus (PCMV), porcine circovirus (PCV), porcine lymphotropic herpesvirus (PLHV), encephalomyocarditis virus (EMCV), and porcine hepatitis E virus. Preferably such a multi-level virological screening strategy undertaken by us as part of our process includes:
Routine monitoring of the herd for the presence of the above viruses.
Routine testing of the donor age group (1-week-old neonates) for the presence of viruses.
Routine testing of the islet and Sertoli cells that are to be used for xenotransplantation.
   Preclinical Studies with Islet-Sertoli Cell Aggregates

In a study conducted at Diatranz's laboratories, the efficacy and safety of transplants of alginate-encapsulated islet-Sertoli cell aggregates (ratio 1:4000) and alginate-encapsulated islets without Sertoli cells were compared in New Zealand white rabbits with experimentally-induced diabetes (5 animals per group).

Both groups received islet cell doses of 10,000 IEQ/kg via intraperitoneal injection. The weekly average blood glucose level declined in both groups over a follow-up period of 5 weeks post-transplantation, and two rabbits in each group were considered to have responded successfully to the transplants. At subsequent postmortem examinations, no abnormal histological findings were found in abdominal organs of recipient animals in either group.

Similar results were achieved in a study of NOD (nonobese diabetic) mice that received intraperitoneal transplants of alginate-encapsulated islets in a dose of 10,000 IEQ/kg with or without Sertoli cells. Two of 5 mice that received islet Sertoli cell aggregates (ratio 1:4000) and 2 of 6 that received islets alone had a partial response, with one animal in each group exhibiting a normal blood glucose level for up to 5 weeks.

Although the ratio of 1:4,000 has been used in these studies it will be clear to those skilled in the art that other ratios may be used without departing from the scope of the invention.

Clinical Studies with Islet-Sertoli Cell Aggregates

We have conducted a number of clinical investigations for our islet-Sertoli cell aggregates. In an experiment islet-Sertoli cell aggregates were transplanted into 12 adolescent type 1 diabetics via the use of subcutaneous stainless steel implant devices that create (on surgical removal of the Teflon® rod) vascularised collagen reservoirs in which the introduced cells are mechanically protected by a steel mesh tube. Initially, two such vascularised collagen reservoirs were created on the upper abdominal wall of each patient, followed by a further two, six months later. Each patient received islet-Sertoli cell aggregates (in ratios varying from 1:30 to 1:100) corresponding to a dose of 250,000 islet equivalents (IEQs) injected into each reservoir, and this dose was repeated in each of the second two reservoirs after 6 months.

Five of the 12 patients responded favourably to this treatment. After a lag period of approximately 8 weeks, the insulin requirements of the 5 patients began to decline and usually fell further after the second transplant. Reductions in the average daily insulin dose of more than 50% were achieved after 12 months, and one patient required no insulin after this time. Improvements in mean daily blood glucose levels and in glycosylated haemoglobin (HbA_{1C}) were also recorded. No evidence of adverse effects were detected in any of the 12 patients, and PERV monitoring tests remained negative after 12 months.

1. A method of preparing aggregates of porcine pancreatic islets and porcine Sertoli cells capable upon implantation into a recipient, of producing insulin in vivo, including or comprising the steps of:
1) isolating porcine islet cells from the pancreas of donor piglets,
2) isolating porcine Sertoli cells from the testes of donor piglets,
3) culturing the Sertoli cells for at least 1 day;
4) adding the isolated porcine islet cells to the cultured Sertoli cells at a predetermined ratio;
5) co-culturing the islet cells and Sertoli cells for at least 1 day;
6) scraping the Sertoli cell layer over the islets to form aggregates; and
7) culturing the aggregates for up to 24 hours.

2. The method of claim 1, wherein said aggregate is a combination of islet:sertoli cells in a predetermined ratio of from about 1:20,000 to about 1:100.

3. The method of claim 2, wherein said ratio is between about 1:2,000 and about 1:4,000.

4. The method of claim 1 wherein said co-culturing step 5) is over a time period of from between about 3 and about 7 days.

5. The method of claim 4, wherein said time period is about 5 days.

6. The method of claim 1, wherein said isolation of the islets is followed by purification of the islets.

7. The method of claim 6, wherein the isolation and purification of the islets together comprise the steps of:
a) surgical removal;
b) collagenase digestion; and
c) washing and culturing of the islets.

8. The method of claim 7, wherein said collagenase digestion involves Liberase H and Xylocaine.

9. The method of claim 1, wherein said isolation of the Sertoli cells is followed by purification of the Sertoli cells.

10. The method of claim 9, wherein said isolation and purification of the Sertoli cells together comprise or include the steps of:
a) surgical removal,
b) digestion with trypsin and Dnase; and
c) washing and culturing of said cells.

11. The method of claim 1, wherein the method further includes the additional step of:
8) virological and microbiological testing or monitoring of said aggregates or components thereof.

12. The method of claim 1, wherein the method additionally or alternatively includes a pre-step before step 1) that comprises virological monitoring or testing of one or both of said islets or said Sertoli cells.

13. The method of claim 1, wherein the method additionally or alternatively includes a pre-step before step 1) of virological monitoring or testing of the piglet donors.

14. The method of claim 1, wherein said islets and Sertoli cells are derived from the same herd or from the same donor piglet(s).

15. The method of claim 14, wherein said donor piglet(s) are about one week old donors.

16. The method of claim 14, wherein said donor piglet(s) are monitored or tested for infectious agents.

17. The method of claim 14, wherein said donor piglet(s) are from a New Zealand pig herd.

18. The method of claim 1, wherein the step of the formation of the aggregate additionally or alternatively comprises the preservation of the original characteristics or native structure of the islets.

19. An aggregate of porcine islets with Sertoli cells prepared substantially according to the method of claim 1.

20. A method of treating a patient suffering from diabetes mellitus comprising the steps of:
1) preparing one or more aggregates of porcine islets with Sertoli cells substantially according to the method of claim 1; and
2) implanting or otherwise administering one or more of said aggregates to said patient.

21. The method of claim 20, wherein said step of implanting or administering the aggregate may be by:
(a) encapsulation of the aggregate in a suitable biocompatible material;
(b) confinement into a suitable device;
(c) inclusion in a matrix that comprises gelatin, collagen or natural carbohydrate polymers, or
(d) inclusion in a plasma thrombin clot or an autologous plasma clot produced with allogeneic thrombin.

22. The method of claim 21, wherein said biocompatible material comprises a suitable alginate.

23. The method of claim 21, wherein said suitable device is a vascularized tube.

24. A device for implantation into a recipient suffering from diabetes mellitus, wherein said device comprises an aggregate of porcine islets with Sertoli cells prepared substantially according to the method of claim 1.

25. The device of claim 24, wherein said device incorporating the aggregates may be one of:
(a) suitable biocompatible material as a capsule;
(b) a vascularized tube;
(c) a matrix preparation comprising gelatin, collagen, or natural carbohydrate polymers: or
(d) a plasma thrombin clot or an autologous plasma clot produced with allogeneic thrombin.

26. The device of claim 25, wherein said biocompatible material comprises a suitable alginate.

27-28. (canceled)
